(19) United States
(12) Patent Application Publication
(10) Pub. No.: US 2005/0182466 A1
(43) Pub. Date: Aug. 18, 2005

(54) Catheter and cable inspection system and method

(76) Inventor: Atul Mahajan, Tempe, AZ (US)

Correspondence Address: SNELL & WILMER;ONE ARIZONA CENTER, 400 EAST VAN BUREN, PHOENIX, AZ, 850040001, US

(21) Appl. No.: 10/963,221

(22) Filed: Oct. 12, 2004

Related U.S. Application Data
(60) Provisional application No. 60/510,462, filed on Oct. 10, 2003.

(51) Int. Cl. A61N 1/00; A61N 1/04; A61N 1/05; A61N 1/06

(52) U.S. Cl. 607/116; 607/122

(57) ABSTRACT

The invention includes an automated inspection system, which automates the evaluation of electrical integrity to determine whether electrode catheters and/or cables are functioning properly. An exemplary embodiment of the system is to provide a system and method for automating the testing of various electrical parameters of several electrode catheters and/or cables so that its performance can be evaluated. The automated inspection system also automates, for example, catheter/cable identification, analysis to known specifications, and report printing and recording of the data analyses.

### CROSS REFERENCE TO RELATED APPLICATION

This application claims the benefit of, and priority to, provisional application Ser. No. 60/510,462, filed Oct. 10, 2003, which application is hereby incorporated by reference in its entirety.

### FIELD OF THE INVENTION

The present invention relates to automated test equipment, including computerized equipment for testing electrode catheters and cables (e.g. electrophysiology diagnostic or therapeutic catheters, anatomic signal recording devices, electrocoagulation or electrocautery devices and cables, or the like). More specifically, this invention pertains to an automated, high volume process for detecting, identifying and quantifying electrical viability of reprocessed electro-physiology catheters and cables.

### BACKGROUND OF THE INVENTION

Electro-physiology catheters and other electrical transmission devices typically consist of a central core of wires terminating at one end with one or more electrodes, and at the opposite end with connector pins. The end of the electrode catheter having the electrodes often is configured with a low profile to be readily inserted into the blood vessel or other body cavity. The central core of wires that comprise the electrode catheter terminate at the other end typically with a pin plug or other electrical connector which can be attached to or coupled with an electronic instrument for pacing or for recording electrical propagation information.

The present invention relates to automated testing configured for testing of electrophysiology catheters/cables used for in vivo studying of the electrical activities of the heart. Typically, catheters of this type contain a plurality of surface electrodes, which are of sufficient conductivity that, when inserted into the heart through a vein, the electrical activities of the heart may be remotely monitored through electrical leads extending from the catheter electrodes through the vein and externally of the subject. Catheters of this type are frequently inserted in a patient experiencing problems with heart function; and, the recordings of the heart electrical activity, as sensed by the catheter electrodes, have proven to be very helpful to medical practitioners in evaluating abnormalities and malfunction of the heart.

Electrophysiology catheters are often designed with one or more electrodes at the distal end of the catheter and are generally independently wired so that each electrode will have an independent connector at the handle end of the catheter. Alternatively, each of the electrodes can have its own conducting wire terminate at the handle in one or more multi-pin connector. In the latter case, the connector is usually designed to connect to a connecting cable, which then has independent connectors at its opposite end. Most of these catheters connect to hardware devices that are some distance from the patient in which the catheter is used, so the connecting cable is desirable.

Many studies have shown that two primary considerations in the inspection of EP catheters and cables are cleaning and sterilization to prevent or minimize the risk of spreading disease-bearing microbes, and the functional reliability of the catheter following repeated use. Sterilization just prior to re-use eliminates or minimizes the catheter/cable microbes, whereupon when properly and rigorously cleaned, re-use of catheters and cables did not create an obvious increase in the risk of infection. However, it has also been found that repeated sterilization may make the catheter and its electronic components fragile, thereby possibly leading to the deterioration of the mechanical and electrical properties of the catheter.

Currently, catheters/cables are subject to several checks to determine their functional reliability. For example, a visual check is made to determine if any part of the catheter is cracked or too stiff to be used properly. This visual check will also reveal any outward physical damage to the tip or the electrodes of the catheter. Also, records are currently maintained on the number of times a catheter/cable has been re-used, and limits are usually placed on the maximum number of uses of a particular catheter/cable.

As part of determining the functional reliability of the catheter prior to its sterilization, catheters presently are often tested for electrical continuity, wherein a standard ohmmeter is used manually to measure resistance. This test typically includes, for example, a technician to hold one probe of the ohmmeter against a socket or wire at the connective end of the catheter, and the other probe against one of the electrodes at the electrode end of the catheter. The probe used at the electrode end of the catheter is brought into contact with corresponding pin of the connector at the other end. If there is continuity between the electrode and the connector pin, the ohmmeter will display the resistance value indicating electrical continuity in that wire. This test procedure is repeated for each wire in the catheter.

The above described test procedure usually includes both hands to adequately hold the probes in place and to manipulate the probes properly, and becomes rather cumbersome to carry out testing adequately. Also, when attempted manually, the probes may not make proper contact with the elements against which they are placed, possibly resulting in false positive or negative readings. A false negative reading can result in discarding a perfectly functional catheter. A false positive reading can result in inserting a defective catheter into the artery of a patient. The situation may be further complicated by the variety of catheters commercially available to medical practitioners and of the variations in electrode size, spacing and characteristics.

Thus, it has long been desired to provide a simple, reliable and easy to use system or method of quickly performing the electrical tests on a heart catheter and cable to demonstrate its proper functioning. It has further been desired to provide such a system or method to automate this rapid testing in a way, which is reliable, efficient and not complex so that a large number of catheters/cables can be tested in less time.

### SUMMARY OF THE INVENTION

The present invention includes an exemplary system which facilitates automation of a practical electrical continuity test to determine the extent of functionality of an EP catheter and/or cable. An exemplary embodiment of the system is to provide a system for testing the electrode catheter/cable so that its functionality can be determined for making multiple uses of the catheter/cable possible. The system of the present invention utilizes, for example, a computer, an electrical resistance measuring device, switching hardware, software program, Junction hardware and a novel support structure for providing common connection between one of the probes of the measuring device and multiple catheters having variable number of electrodes with variable electrode pacing.

An exemplary embodiment of the present invention is provided in an EP-Catheter automated testing system including a support structures and interfaces as test fixtures for holding catheters and cables under test. The first probe of an electrical resistance measuring device couples or connects to the connector sockets via the support structure having electrical contact elements configured to removably receive the connective end of a cardiac catheter, where the catheter has at least one signal generating electrode at an end of the catheter substantially opposite the connective end of the catheter. Electrical contact elements are electrically coupled to the receptacles to form an electrical path between the first probe inserted in one of the receptacles, the corresponding electrical contact element, and the signal-generating electrode. A second probe electrically coupled to the other terminal of the electrical resistance-measuring device is coupled to another support structure. This support structure houses the electrode ends of catheters under test. The measuring device along with a hardware system measures the electrical resistance when it is instructed programmatically. The automated inspection system controls the electrical resistance measuring device and a multiplexing unit to substantially sequentially connect each electrode under test, measure the resistance along the electrode circuit for continuity testing or between electrodes for insulation testing, and records resistance values. The automated inspection system then analyses these values for all catheters and provides test results statistics. It saves the data for manual inspection and provides test reports in any form known in the art, such as, for example, it prints the test reports on default printer.

### BRIEF DESCRIPTION OF THE DRAWINGS

This invention will be better understood from the following description taken in conjunction with the accompanying drawings. Each of the following figures diagrammatically illustrates aspects of the present invention. The illustrations provide examples of the invention described herein. Like elements in the various figures often are represented by identical numbering.

FIG. 1 shows an exemplary flow chart of prior art for inspecting catheters.

FIG. 2 shows a perspective view of an exemplary prior art device for inspecting catheters.

FIG. 3 shows a general block diagram of exemplary automated inspection equipment of the invention.

FIG. 4 shows a perspective view of an exemplary junction box of the invention.

FIG. 5 shows a schematic view of exemplary automated inspection equipment.

FIG. 6 shows a block diagram of an exemplary automated inspection equipment of the invention.

FIG. 7 shows a flow chart of an exemplary method to perform the automated processes for the automated inspection equipment of the invention.

FIG. 8 shows exemplary flow chart breakout #1 of the flow chart in FIG. 7.

FIG. 9 shows exemplary flow chart breakout #2 of the flow chart in FIG. 7.

FIG. 10 shows exemplary flow chart breakout #3 of the flow chart in FIG. 7.

FIG. 11 shows exemplary flow chart breakout #4 of the flow chart in FIG. 7.

FIG. 12 shows exemplary flow chart breakout #5 of the flow chart in FIG. 7.

FIG. 13 shows a perspective view of an exemplary common reference box of the invention.

FIGS. 14A to 14C shows exemplary components of the common reference box.

FIGS. 15A and 15B show a perspective view and a side view of an alternative common reference box.

FIGS. 16A to 16G show a top view, three sectional views, and three detail views of an exemplary common reference box frame in FIGS. 15A and 15B.

### DETAILED DESCRIPTION OF THE INVENTION

The detailed description of exemplary embodiments of the invention herein makes reference to the accompanying drawings and pictures, which show an exemplary embodiment by way of illustration and its best mode. While these exemplary embodiments are described in sufficient detail to enable those skilled in the art to practice the invention, it should be understood that other embodiments may be realized and that logical and mechanical changes may be made without departing from the spirit and scope of the invention. Thus, the detailed description herein is presented for purposes of illustration only and not of limitation. For example, the steps recited in any of the method or process descriptions may be executed in any order and are not limited to the order presented.

An exemplary embodiment of the present invention is to provide an automated testing system for testing of electrophysiology catheters/cables and facilitate a technician to rapidly and accurately test a large number of catheters/cables for electrical continuity. Another exemplary embodiment of the present invention is to provide a complete testing system for cardiac catheters and cables, which automatically measures, interprets, logs the test data, and prints test reports. Another exemplary embodiment of the present invention is to provide an interface for connecting a large of number of catheters/cables to the testing system and positively maintains a connection between the connective ends of all catheters under test.

Another exemplary embodiment of the present invention is to provide a support structure, which provides an interface for connecting electrode ends of all catheters under test to the testing system. A further exemplary embodiment of the present invention is to provide a support structure, which does not damage catheters or cables mechanically or electrically. An additional exemplary embodiment of the present invention is to provide a multi-catheter/cable EP automated testing system having support structures which forms an electrical contact path between each of the connective end sockets of the catheters and to all eletrctode rings at the electrode end of catheters. Even though there are multiple electrodes coupled together, the Test system isolates and measure continuity of a single catheter electrode or insulation between any two adjacent catheter electrodes.

The embodiments of the invention enable automated inspection of a variety of electrical parameters for catheters containing electrodes, wherein the electrodes may be routed to connector pins at a handle. The embodiments of the invention also enable automated inspection of cables used to transmit signals from the catheter electrodes to monitoring equipment. The present invention variously includes the devices as well as the methodology disclosed. Furthermore, it is contemplated that sub-combinations of features, especially of the connector and coupling features disclosed, comprise aspects of the invention.

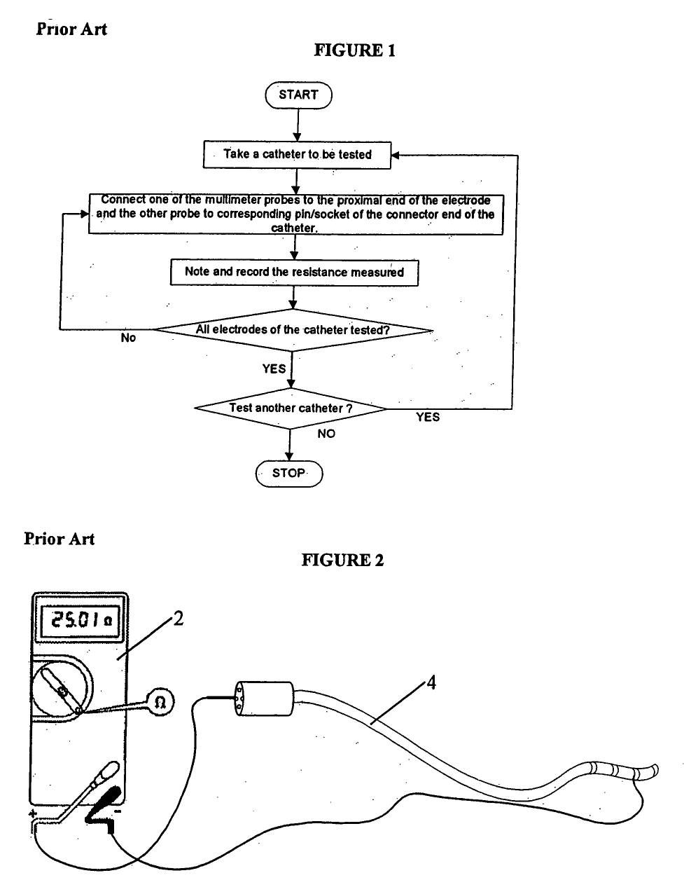

FIG. 1 shows an exemplary flow chart for testing the electrical integrity of electrode catheters manually, indicative of the prior art. A single catheter to be tested is chosen. One pole of a multimeter 2, as shown in FIG. 2, is coupled to one connector pin at the handle of the catheter 4 while the remaining multimeter pole is used to engage the electrode on the distal end of the catheter. The multimeter displays the resistance. A measured value is compared to a high threshold for that specific catheter design and the electrode may be classified as functional if the resistance is less than the high threshold; otherwise, the catheter may be rejected. After all catheter electrodes are tested for continuity, then insulation is tested by placing one pole of the multimeter on one connector pin at the handle and engaging the remaining multimeter pole to a second connector pin at the handle. The electrodes should be positioned so that they do not contact conductive surfaces, which could interfere with insulation testing.

The multimeter is used to determine the resistance between electrode signal wires. Interelectrode resistance is then compared to a lower threshold, e.g. 1 Megaohm for a d.c. measurement, for each combination of electrodes to ensure electrical signals do not communicate between electrode circuits. This process is extremely time-consuming and operator dependent, especially since the connector pins and electrodes are extremely small compared to the multimeter probes. As such, the invention describes embodiments to automate some or all aspects of electrical integrity testing.

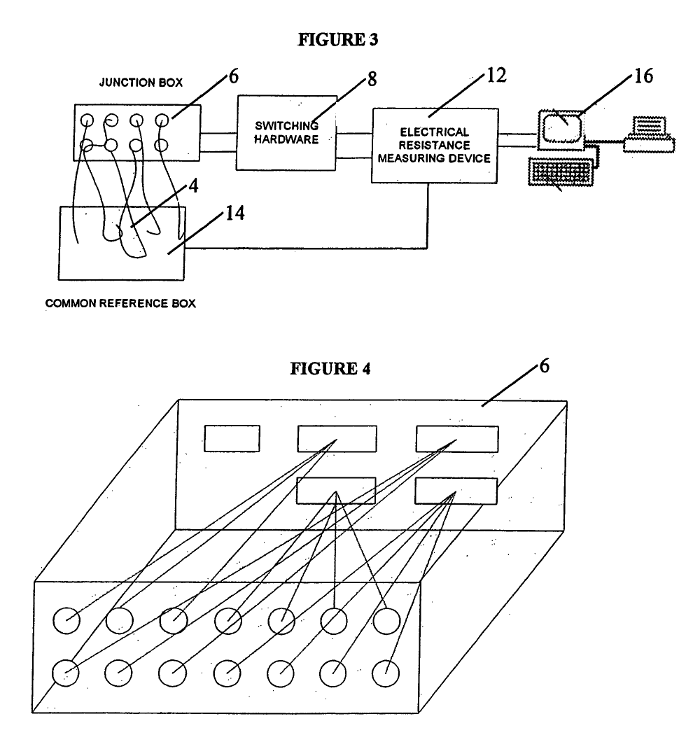

FIG. 3 shows a block diagram of an exemplary automated inspection system 10 of the invention. As shown in FIG. 3, a junction box 6, to which several electrode catheters 4, or cables, may be coupled, is routed through a multiplexer 8 to a resistance (or impedance) measuring device 12. The common reference box 14 is also routed to the electrical resistance-measuring device 12. The common reference box 14 is used to couple all catheter electrodes 4 together for measuring the resistance between the electrodes and each connector pin to substantially ensure continuity from each connector pin to each electrode. A computer 16 with certain software may be used to control the multiplexer 8 and the electrical resistance-measuring device 12 to sequence through all electrodes for continuity testing and electrode pairs for insulation testing. It should be noted that alternate inspection parameters can be automated using the automated inspection system of the invention including, for example, 1) dielectric insulation between electrode circuits, 2) dielectric testing between electrode circuits and the exterior surface of the catheter, 3) leakage current between electrodes, 4) pacing stimulus efficiency, 5) signal to noise ratio, etc.

FIG. 4 shows an exemplary junction box of the invention that routes the connectors of multiple catheters, or cables, to the multiplexer.

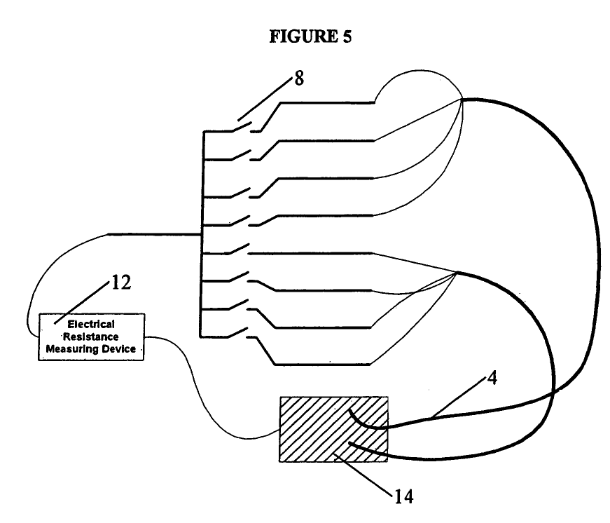

FIG. 5 shows a schematic of an exemplary automated inspection system of the invention. The catheters, or cables, are coupled to the junction boxes at the handles of the catheters, or cables. The junction boxes are coupled to the multiplexing unit, which is controlled by the computer. The electrode ends of the catheters are engaged to the common reference box during continuity testing. For cables, the opposite end is coupled to another junction box that functions as the common reference box. During insulation testing between electrode pairs, the catheters, or cables, are disengaged from the common reference box.

FIG. 6 shows a block diagram for another embodiment of the invention. Multiple catheter and cable configurations are coupled to dedicated junction boxes, which are routed to a branching box 18. The branching box 18 is coupled to the multiplexing unit, which may be controlled by the computer. The switching modules of the multiplexing unit are used to connect various catheter electrode circuits to the electrical integrity-measuring device incorporated in the computer as a module or as an external device.

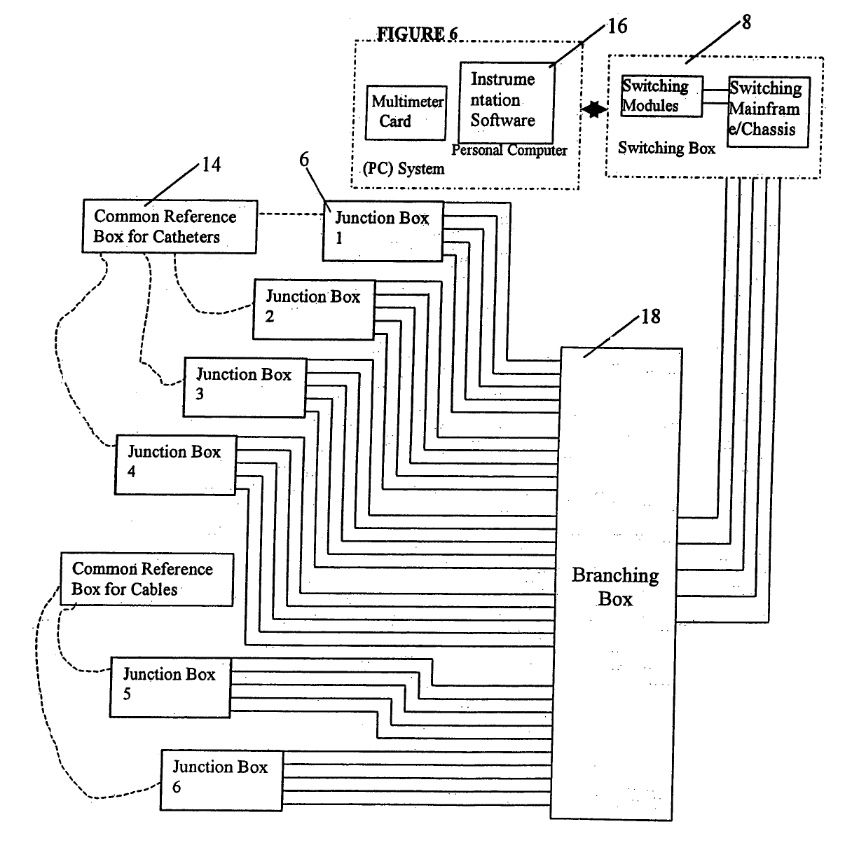

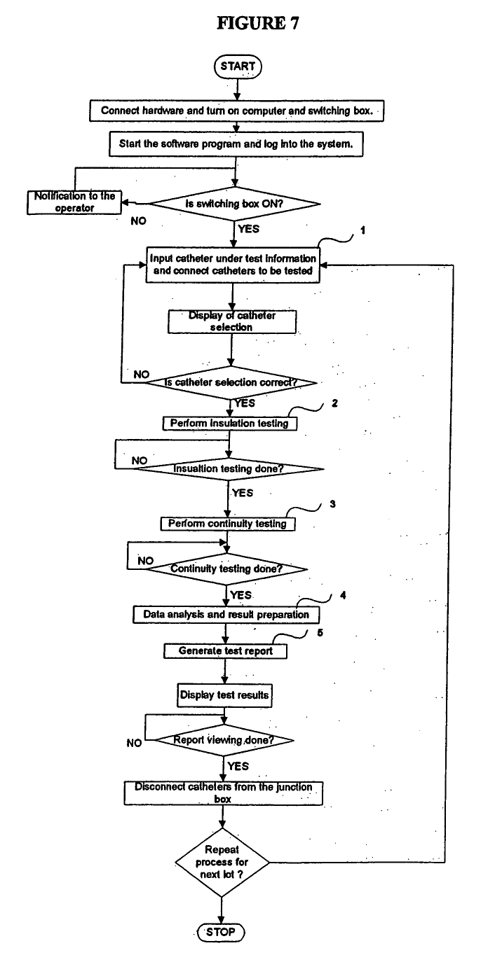

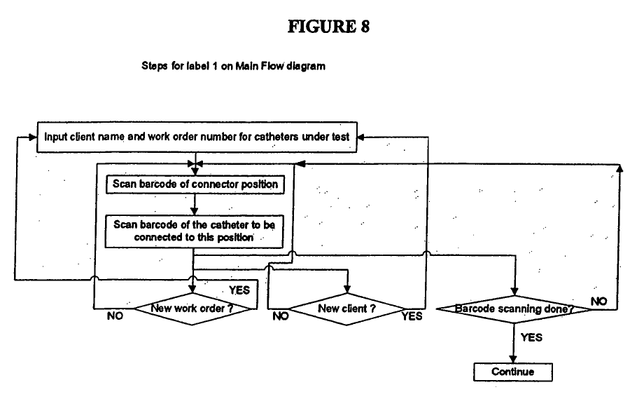

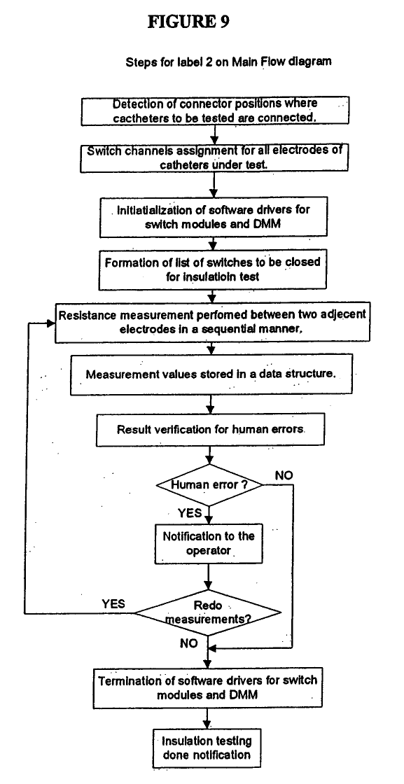

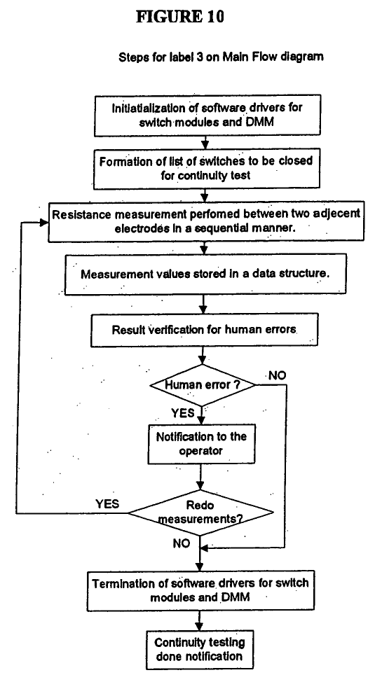

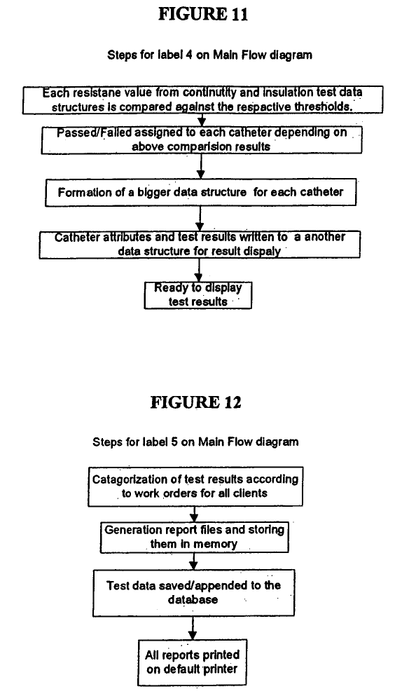

FIG. 7 shows a flow chart of an exemplary method used to control the resistance-measuring device and multiplexing unit to facilitate automating electrical continuity and insulation testing by the automated inspection system. FIGS. 8 to 12 show exemplary flow chart breakouts for points 1 to 5 in the flow chart in FIG. 7. The automated inspection system software performs, for example, device logging functions, measuring functions, data deciphering functions (e.g. comparing measured values to thresholds), and data recording.

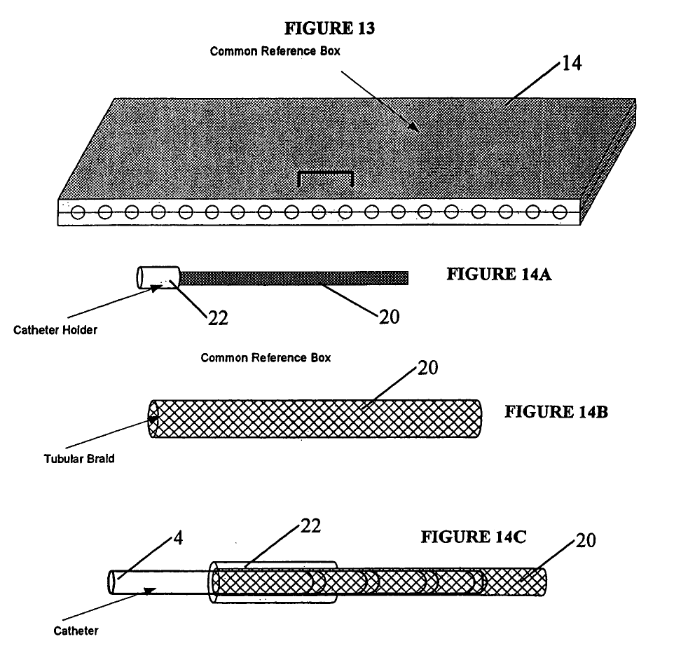

FIG. 13 shows a perspective view of an exemplary common reference box 14 used to couple some or all electrodes of the catheters 4 to the resistance-measuring device 12. FIGS. 14A and 14B show exemplary conductive components of the common reference box 14 to couple the electrodes of the catheters to the common reference box. FIG. 14A shows a catheter port 22 with a braided tube 20, also shown in FIG. 14B. Each catheter is inserted through the port and into the braided tube, as shown in FIG. 14C. Once in the tube, the top of the common reference box is closed against the bottom slightly compressing the catheter against the braid tube and producing an electrical connection between the catheter electrodes and the conductive braid tube. The conductive braid tubes are, for example, soldered, spot welded, or otherwise bonded together such that they are electrically coupled together. The common reference box couples the conductive braid tubes to the resistance-measuring device.

For cables, the common reference box is simplified since the opposite ends of the cables are coupled to another junction box that interconnects some or all pins of the cable connector together and routes that connection to the resistance measuring device.

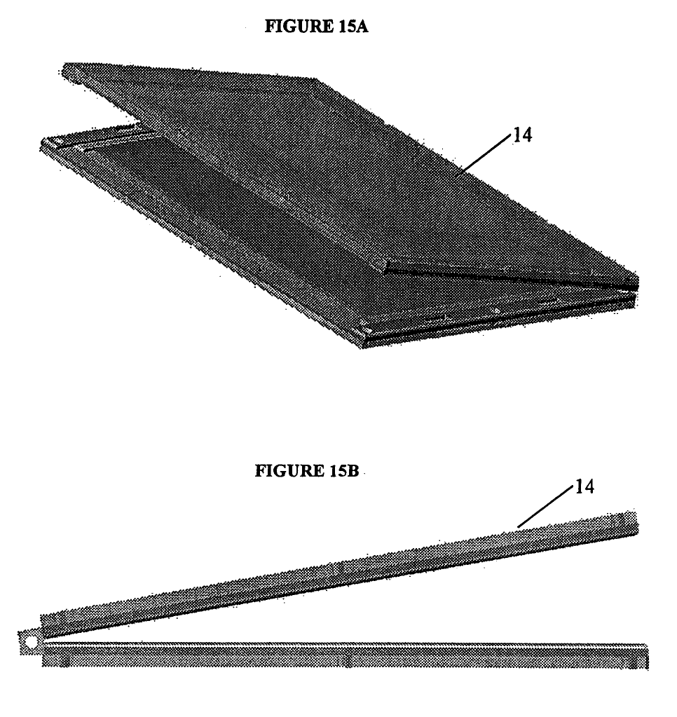

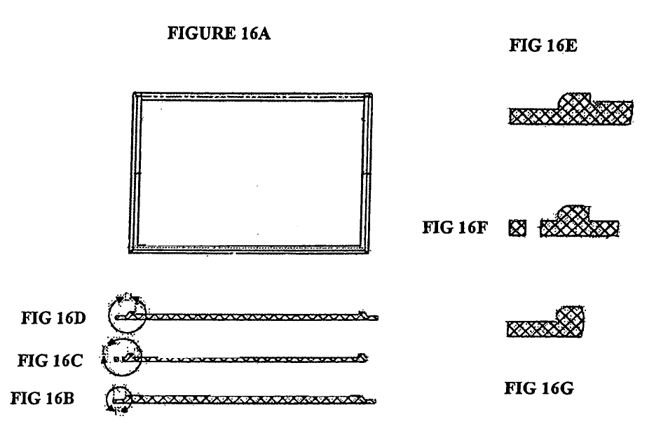

FIGS. 15A and 15B show a perspective view and a side view of an alternative common reference box. A bottom and a top frame, as shown in FIGS. 16A to 16G are oriented into, for example, a clamshell orientation such that the top frame rotates relative to the bottom frame at a hinge. The frames connect to surfaces in which a compressible sheet is attached. This compressible sheet can be fabricated from, for example, silicone, urethane, nylon, or other biocompatible material that has suitable elastic recoil to resist or substantially withstand compression with minimal permanently deformation. Sheets of braided wire, conductive polymer, conductive foam, or other material that is conductive but is capable of molding to the catheter surface, are secured to the top and bottom frames. The catheters are placed flat in the common reference box such that when the top and bottom frames are closed, the catheter electrodes are compressed against the conductive sheets such that the electrodes are electrically coupled to the common reference box which is itself coupled to the resistance measuring device.

The invention has been described and specific examples or variations of the invention have been portrayed. The use of those specific examples is not intended to limit the invention in any way. In all, it is to be understood that each of the features described in connection with the various connector components and projections for forming the same may be mixed and matched to form any number of desirable combinations. Further, it is contemplated that additional details as to the use or other aspects of the system described herein may be drawn from Abstract, Field of the Invention, Background of the Invention, Summary of the Invention, Brief Description of the Drawings, the Drawings themselves and Detailed Description and other background that is intended to form part of the present invention, including any of the patent applications cited above, each of which being incorporated by reference herein in its entirety for any purpose. All equivalents are considered to be within the scope of the claimed invention, even those which may not have been set forth herein merely for the sake of relative brevity. It is also contemplated that any single feature or any combination of optional features of the inventive variations described herein may be specifically excluded from the invention.

As will be appreciated by one of ordinary skill in the art, certain features or functions of the present invention may be embodied as a method, a data processing system, a device for data processing, and/or a computer program product. Accordingly, the present invention may take the form of an entirely software embodiment, an entirely hardware embodiment, or an embodiment combining aspects of both software and hardware. Furthermore, the present invention may take the form of a computer program product on a computer-readable storage medium having computer-readable program code means embodied in the storage medium. Any suitable computer-readable storage medium may be utilized, including hard disks, CD-ROM, optical storage devices, magnetic storage devices, and/or the like.

The present invention is described herein with reference to screen shots, block diagrams and flowchart illustrations of methods, apparatus (e.g., systems), and computer program products according to various aspects of the invention. It will be understood that each functional block of the block diagrams and the flowchart illustrations, and combinations of functional blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including instruction means which implement the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions specified in the flowchart block or blocks.

Accordingly, functional blocks of the block diagrams and flowchart illustrations support combinations of means for performing the specified functions, combinations of steps for performing the specified functions, and program instruction means for performing the specified functions. It will also be understood that each functional block of the block diagrams and flowchart illustrations, and combinations of functional blocks in the block diagrams and flowchart illustrations can be implemented by either special purpose hardware-based computer systems which perform the specified functions or steps, or suitable combinations of special purpose hardware and computer instructions.

Benefits, other advantages, and solutions to problems have been described herein with regard to specific embodiments. However, the benefits, advantages, solutions to problems, and any element(s) that may cause any benefit, advantage, or solution to occur or become more pronounced are not to be construed as critical, required, or essential features or elements of any or all the claims or the invention. As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus. Further, no element described herein is required for the practice of the invention unless expressly described as “essential” or “critical”.

1. A system for testing the functional reliability of electrophysiology catheters and cables comprising:
an electrical resistance measuring device;
a first support structure, having electrical contact elements for receiving a connective end of one or more catheters, connected to the electrical resistance measuring device; and
a second support structure for receiving an electrode end of one or more catheters connected to the electrical resistance measuring device.

2. The system of claim 1 further comprising switching hardware connected to the second support structure and the electrical resistance measuring device.

3. The system of claim 2 wherein the switching hardware comprises a multiplexer.

4. The system of claim 2 further comprising a computer for controlling the switching hardware.

5. The system of claim 2 further comprising a display for viewing test data or test reports.

6. A method for automatically testing the functional reliability of one or more electrophysiology catheters and cables comprising the steps of:
connecting a connective end of one or more catheters to a first support structure having electrical contact elements for receiving the connective end of one or more catheters;
connecting an electrode end of one or more catheters to a second support structure;
connecting the first and second support structures to an electrical resistance measuring device;
providing a switching hardware between the second support structure and the electrical resistance measuring device; and
programming the electrical resistance measuring device and the switching hardware to sequentially connect each electrode to measure at least one of electrode circuit continuity and insulation between electrodes.

7. The method of claim 6 further comprising the step of recording the measurements obtained from programming the electrical resistance measuring device and the switching hardware.

8. The method of claim 7 further comprising the step of displaying the recorded measurements on at least one of a monitor and a written report.

9. The method of claim 6 wherein the step of providing a switching hardware comprises the step of providing a multiplexer between the second support structure and the electrical resistance measuring device.

10. The method of claim 6 wherein the step of connecting the first and second support structures to an electrical resistance measuring device comprises the step of connecting the first support structure to a first probe embodying part of the electrical resistance measuring device and connecting the second support structure to a second probe embodying part of the electrical resistance measuring device.
